(19) United States
(12) Patent Application Publication
(10) Pub. No.: US 2024/0084428 A1
(43) Pub. Date: Mar. 14, 2024

(54) TISSUE EXERCISING AND STIMULATING BIOREACTOR

(71) Applicant: Brian Joseph Stasey, Fishers, IN (US)

(72) Inventor: Brian Joseph Stasey, Fishers, IN (US)

(21) Appl. No.: 18/078,122

(22) Filed: Dec. 9, 2022

Related U.S. Application Data
(60) Provisional application No. 63/405,798, filed on Sep. 12, 2022.

(51) Int. Cl. C22F 1/00; F03G 7/06

(52) U.S. Cl. CPC C22F 1/006; F03G 7/06

(57) ABSTRACT

In one embodiment, a tissue bioreactor is provided including a basin, a first elongated element, and a second elongated element. The basin includes a base and a sidewall. The base and the sidewall define a chamber adapted to contain a tissue sample. The first elongated element extends into the chamber of the basin. The first elongated element is bio-compatible and is electrically conductive. The second elongated element also extends into the chamber of the basin. The second elongated element is bio-compatible and is spaced apart from the first elongated element within the chamber.

### TECHNICAL FIELD

This disclosure relates to an apparatus for growing, exercising, stimulating, maturing, and testing biological tissue, and in particular this disclosure is related to an apparatus for growing and testing muscular tissue.

### BACKGROUND

The statements in this section merely provide background information related to the present disclosure and may not constitute prior art.

The number of pharmaceuticals that must be tested for efficacy keeps increasing, especially as the population ages. There is a need for rapid methods to screen potential pharmaceuticals and identify leading pharmaceutical candidates. It is not practical to test every candidate on a human or even an animal subject. It is much more fruitful to use human tissue in multiple bioreactors in order to be able to test many different drug candidates on the same tissue. This also applies to personalized medicine where a single person's tissues can be cloned, and then many drugs can be tested in vitro for candidates that may be most efficacious for that individual. Bioreactors may be used in a variety of applications that include drug testing, tissue repair, tissue replacement, treatment, regenerative medicine, or combinations thereof.

Tissue engineering seeks to develop biological substitutes that restore, maintain, or improve tissue function. A major challenge is the need to get engineered tissues to be more like native tissues. These tissues are grown in bioreactors that mimic the conditions inside the body that the tissue is accustomed to. The more closely these tissues can be grown in vitro to conditions the tissues would experience in the body, the more closely they can match normal bodily tissues. And thus, the more likely that drugs tested on these tissues in vitro will act the same in the body.

Tissue engineering methods currently allow for muscular tissue (such as heart tissue) to be grown in vitro and allow for the tissue to beat (exercise) against a resistance in order to get stronger and mature from fetal heart tissue to infant heart tissue.

In particular, growing and testing muscular tissue generally requires (1) that the tissue be grown around a flexible testing device, (2) that the tissue be electrically stimulated to induce contractions, and (3) that the contractions of the muscular tissue are measured. For example, in some embodiments, the tissue may be grown around a pair of flexible rods anchored to a basin. Once the tissue has grown around flexible rods, electrodes may be inserted into the muscular tissue to deliver electrical stimulation. The contraction of the muscular tissue may then be measured optically.

Such an embodiment has numerous constraints and difficulties. For example: (1) tissue must typically grow around bio-compatible material, severely constraining the types of materials that may be used for the flexible rods; (2) separate electrodes increases the complexity and cost of the testing apparatus; (3) inserting electrodes into the tissue may damage the tissue or form cavities which may affect the outcome of the testing; and (4) optical measurement of the contraction requires additional human oversight of the apparatus or otherwise costly optical observation and recognition devices and software.

Therefore, it is desirable that a testing apparatus could be provided (1) which is made from a bio-compatible material, (2) which serves as both a flexible measurement device and the electrode, and (3) which would allow additional, more efficient ways of measuring the contraction of the tissue.

### SUMMARY

Further areas of applicability will become apparent from the description provided herein. It should be understood that the description and specific examples are intended for purposes of illustration only and are not intended to limit the scope of the present disclosure.

In one embodiment, a tissue bioreactor is provided including a basin, a first elongated element, and a second elongated element. The basin includes a base and a sidewall. The base and the sidewall define a chamber adapted to contain a tissue sample. The first elongated element extends into the chamber of the basin. The first elongated element is bio-compatible and is electrically conductive. The second elongated element also extends into the chamber of the basin. The second elongated element is bio-compatible and is spaced apart from the first elongated element within the chamber.

In another embodiment, a tissue testing apparatus is provided including a basin, a tissue sample, a first elongated element, and a second elongated element. The basin includes a base and a sidewall. The base and the sidewall define a chamber. The tissue sample is arranged within the chamber of the basin. The first elongated element extends into the chamber of the basin, is bio-compatible, and is also electrically conductive. The second elongated element also extends into the basin and is spaced apart from the first elongated element within the chamber. The second elongated element is bio-compatible and is also electrically conductive.

In yet another embodiment, a method of using a tissue bioreactor is provided. The tissue bioreactor includes a basin, a tissue sample, a first elongated element, and a second elongated element. The basin includes a base and a sidewall defining a chamber. The tissue sample is positioned within the chamber. The first elongated element is coupled to a first side of the tissue sample. The second elongated element is coupled to a second side of the tissue sample. The method includes passing an electrical current through the first elongated element, the tissue sample, and the second elongated element to induce contraction of the tissue sample. The method further includes measuring a deflection of the first elongated element resulting from the contraction of the tissue sample.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a perspective view of a first example of a tissue bioreactor include a basin, two elongated elements, and a tissue sample;

FIG. 2 illustrates a perspective view of a second example of a tissue bioreactor include a basin, two elongated elements, and a tissue sample;

FIG. 3 illustrates a perspective view of a third example of a tissue bioreactor include a basin and two elongated elements;

FIG. 4 illustrates a cross-sectional side view of a fourth example of a tissue bioreactor include a basin and two elongated elements;

FIG. 5 illustrates a cross-sectional side view of a fifth example of a tissue bioreactor include a basin and two elongated elements;

FIG. 6 illustrates a side view of an example of an elongated element;

FIG. 7 illustrates a perspective view of a sixth example of a tissue bioreactor include a basin, two elongated elements, and a tissue sample;

FIG. 8 illustrates another perspective view of the sixth example of a tissue bioreactor include a basin, two elongated elements, and a tissue sample;

FIG. 9 illustrates yet another perspective view of the sixth example of a tissue bioreactor include a basin, two elongated elements, and a tissue sample;

FIG. 10 illustrates a flow diagram of operations for using a tissue bioreactor.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

In one example, a tissue bioreactor is provided including a basin, a first elongated element, and a second elongated element. The basin includes a base and a sidewall. The base and the sidewall define a chamber adapted to contain a tissue sample. The first elongated element extends into the chamber of the basin. The first elongated element is bio-compatible and is electrically conductive. The second elongated element also extends into the chamber of the basin. The second elongated element is bio-compatible and is spaced apart from the first elongated element within the chamber.

One technical advantage of the systems and methods described below may be that combining the supportive element and the electrifying element into a single component may reduce the complexity of the testing apparatus. This may reduce the cost of testing and increase the reliability of the testing apparatus.

Another technical advantage of the systems and methods described below may be that combining the supportive element and the electrifying element into a single component may avoid damaging the tissue sample before electrifying it. For example, no additional electrode would have to be inserted into the tissue sample before testing could begin.

Yet another technical advantage of the systems and methods described below may be that combining the supportive element and the electrifying element into a single component may allow for more efficient methods of measuring the contraction of the tissue. For example, optical observation of the tissue sample may not be needed, requiring fewer resource to operate the testing apparatus.

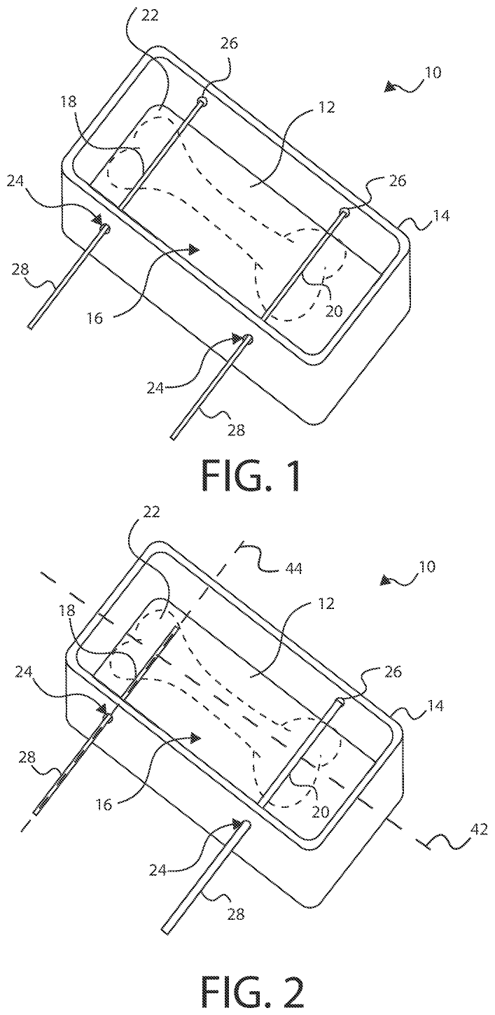

FIG. 1 illustrates a perspective view of an embodiment of a tissue bioreactor including a basin 10, a first elongated element 18, a second elongated element 20, and a tissue sample 22. The basin 10 may be any component which is adapted to contain the tissue sample 22. Examples of the basin 10 may include a bowl, a tub, or a dish. The basin 10 may be made of a non-conductive material to prevent misdirection of electrical signals. Further, the basin 10 may be made of a non-biocompatible material to prevent the tissue sample 22 from securing itself to the basin 10.

The basin 10 may include a base 12 and a sidewall 14 which define a chamber 16 adapted to contain the tissue sample 22. The base 12 may be any component of the basin 10 upon which the tissue sample 22 can be placed. Examples of the base 12 include a plate, a dish, or a bowl. For example, the basin 10 may be 5 inches long, 1 inch wide, and 0.3 inches deep. The sidewall 14 may be any portion of the basin 10 which extends upward from the base 12 and encloses the chamber 16 to contain the tissue sample 22. Examples of the sidewall 14 may include a wall, a lip, or an inclined barrier. In some embodiments, the base 12 and the sidewall 14 may create a curved contoured wall. The basin 10 may also include one or more openings 24 in the sidewall 14 to allow the first elongated element 18 and the second elongated element 20 to extend into the chamber 16.

The first elongated element 18 may be any component which extends into the chamber 16 of the basin 10 and is adapted to be coupled to the tissue sample 22. Examples of the first elongated element 18 may include a wire, a filament, or a rod. The first elongated element 18 may extend into the chamber 16 from the openings 24 in the sidewall 14 or may extend across a top of the sidewall 14. The first elongated element 18 may be rigidly coupled to the sidewall 14 at an anchor point 26. The anchor point 26 may be positioned on an opposing portion of the sidewall 14 from the opening 24.

The second elongated element 20 may be any component which extends into the chamber 16 of the basin 10 and is adapted to be coupled to the tissue sample 22. Examples of the second elongated element 20 may include a wire, a filament, or a rod. The second elongated element 20 may extend into the chamber 16 from the openings 24 in the sidewall 14 or may extend across a top of the sidewall 14. The second elongated element 20 may be rigidly coupled to the sidewall 14 at an anchor point 26. The anchor point 26 may be positioned on an opposing portion of the sidewall 14 from the opening 24. The first elongated element 18 may be positioned apart from the second elongated element 20 such that the tissue sample 22 may be placed between them.

The first elongated element 18 and the second elongated element 20 may each be made from a bio-compatible material to allow the tissue sample 22 to be coupled with the first elongated element 18 and the second elongated element 20. Examples of bio-compatible materials may include a Nickel-Titanium alloys such as Nitinol, as well as Gold, Lead, Titanium, polyurethane, and zirconia. The thickness of the first elongated element 18 and the second elongated element 20 may be between 0.001 inches and 0.1 inches. To ensure sufficient flexibility for testing of the tissue sample 22, in some embodiments, the first elongated element 18 may have a thickness which is no more than 0.004 inches.

Additionally, the first elongated element 18 and the second elongated element 20 may be made of an electrically conductive material to allow electrical signals to pass through the first elongated element 18 or the second elongated element 20 and into the tissue sample 22. Examples of electrically conductive materials may include Gold, Titanium, or a Nickel-Titanium alloy such as Nitinol.

Further, the first elongated element 18 and the second elongated element 20 may be made of a superelastic material to allow the first elongated element 18 and the second elongated element 20 to bend repeatedly from contraction of the tissue sample 22 and then return to their original position once the tissue sample 22 has relaxed. Examples of superelastic materials may include Nickel-Titanium alloys such as Nitinol. The combination of the three features of bio-compatibility, electrical conductivity, and superelasticity may allow for the first elongated element 18 and the second elongated element 20 to provide superior performance above prior art bioreactors.

The tissue sample 22 may be any portion of biological tissue which is adapted to be coupled to the first elongated element 18 and the second elongated element 20 within the chamber 16 of the basin 10. Examples of the tissue sample 22 may include cardiac tissue, muscular tissue, or any other tissue which contracts responsive to an electrical signal. In some embodiments, the tissue sample 22 may be placed within the chamber 16 and may grow to become coupled to the first elongated element 18 and the second elongated element 20. In other embodiments, the tissue sample 22 may be initially coupled to the first elongated element 18 and the second elongated element 20 as the tissue sample is being placed into the chamber 16.

The first elongated element 18 and the second elongated element 20 may be coupled to wires 18 leading away from the basin 10 and to an electrical source (not shown). While the tissue sample 22 is being tested, the first elongated element 18, the tissue sample 22, the second elongated element 20, the wires 28, and the power source (not shown) may form an electrical circuit. Through this circuit, electrical impulses may pass through the tissue sample, causing the tissue sample 22 to alternatingly contract and relax. The length of the contraction of the tissue sample 22 may be measured based on the deflection of the first elongated element 18 and the second elongated element 20 from their initial conditions when the tissue sample 22 is in the relaxed state.

FIG. 2 illustrates a perspective view of another embodiment of the tissue bioreactor. In some embodiments the first elongated element 18 and the second elongated element 20 may be identical. However, in other embodiments, the first elongated element 18 and the second elongated element 20 may differ in significant ways. For example, in some embodiments, the first elongated element 18 may be electrically conductive while the second elongated element 20 may be electrically non-conductive. In such embodiments, the first elongated element 18 may have an electrical supply and return portion.

In other embodiments, it may be desirable to maximize deflection one of the first elongated element 18 and second elongated element 20 while minimizing the deflection of the other of the first elongated element 18 and the second elongated element 20. This may be accomplished in one embodiment by having the first elongated element 18 have an end positioned in the chamber 16 and which is unrestricted by the sidewall 14. In such an embodiment, the second elongated element 20 may be coupled to the anchor point 26, minimizing the deflection of the second elongated element 20 and maximizing the deflection of the first elongated element 18 when the tissue sample 22 contracts.

Similarly, in some embodiments the second elongated element 20 may have a thickness which is greater than a thickness of the first elongated element 18. For example, in some embodiments, the second elongated element 20 may have a thickness of 0.004 inches, while the first elongated element 18 may have a thickness of 0.001 inches. In such an embodiment, the first elongated element 18 may have a propensity to bending which is greater than the second elongated element 20.

As illustrated in FIG. 2, the tissue sample 22 may be arranged within the chamber 16 such that contraction of the tissue sample 22 causes the tissue sample 22 to decrease in length along an axis of contraction 42. The first elongated element 18 may be positioned within the chamber 16 along an axis of deflection 44 which may be used as a reference to measure the deflection of the first elongated element 18 due to contraction. The axis of deflection 44 may be perpendicular to the axis of contraction 42 to maximize the deflection of the first elongated element 18 when contraction of the tissue sample 22 occurs.

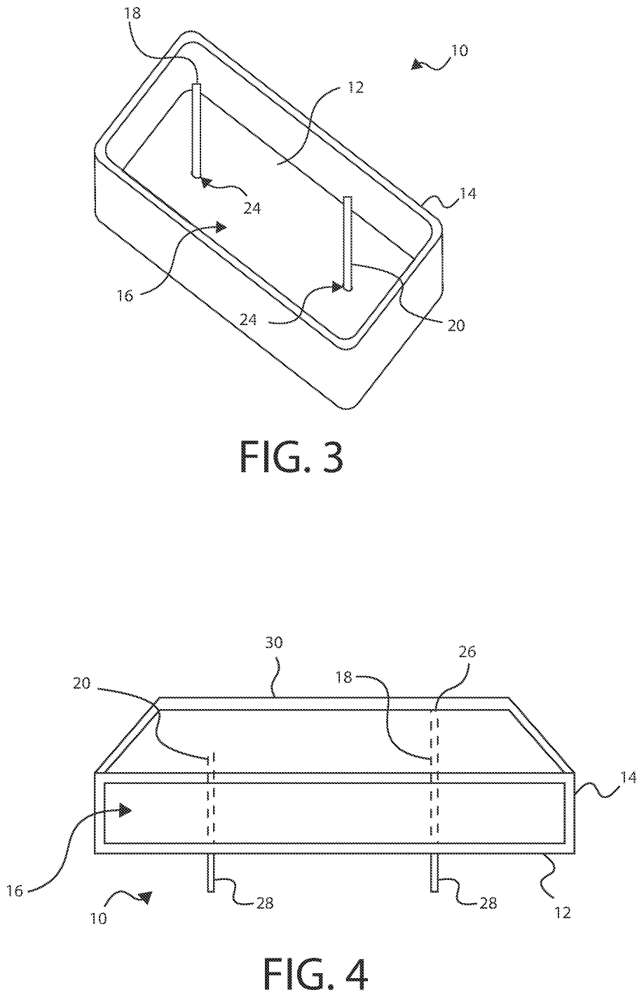

FIG. 3 illustrated a perspective view of yet another embodiment of the tissue bioreactor. In some embodiments, one or more of the first elongated element 18 and the second elongated element 20 may extend into the chamber 16 through the base 12. In such an embodiment, the openings 24 may be situated on the base 12 instead of the sidewall 14 to accommodate the first elongated element 18 and the second elongated element 20. As illustrated in FIG. 3, the ends of the first elongated element 18 and the second elongated element 20 may be positioned within the chamber 16 and may be unrestricted by the base 12, the sidewall 14, or any other structure other than the tissue sample 22.

FIG. 4 shows a side cross-sectional view of yet another embodiment of the tissue bioreactor. In some embodiments, the tissue bioreactor may include a cap 30 coupled to the basin 10. The cap 30 may be any component which is coupled to the basin 10 to cover a portion of the chamber 16 above the sidewall 14. Examples of the cap 30 may include a plate, a grate, or even a supportive beam. In some embodiments, the second elongated element 20 may be coupled to an anchor point 26 on the cap 30, to restrict movement of the second elongated element 20 within the chamber 16.

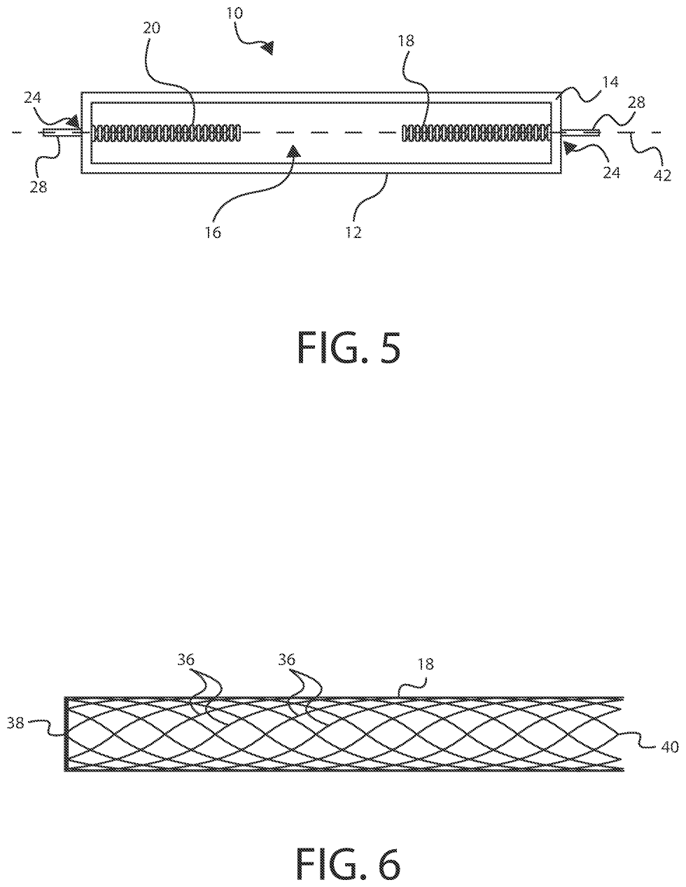

FIG. 5 illustrates a side cross-sectional view of yet another embodiment of the tissue bioreactor. In some embodiments, the first elongated element 18 and the second elongated element 20 may have the form of a coiled spring. The coiled shape of the first elongated element 18 may create more surface area to be coupled to the tissue sample 22, enhancing the connection between the two components. Further, in such embodiments, one or both of the first elongated element 18 and second elongated element 20 may be positioned within the chamber 16 along the axis of contraction 42, such that when the tissue sample 22 contracts, the deflection of the first elongated element 18 occurs as an extension of the coiled spring of the first elongated element 18.

In other embodiments, the first elongated element 18 may be arranged along the axis of deflection 44 perpendicular to the axis of contraction 42 (as shown in FIG. 2). In such embodiments, the coiled spring of the first elongated element 18 may enhance the deflection of the first elongated element 18 making measurement easier. In some embodiments, only one of the first elongated element 18 and the second elongated element 20 may include a coiled spring while the other is a less flexible element. This may enhance the measurability of deflection due to contraction of the tissue sample 22.

FIG. 6 illustrates another possible embodiment of the first elongated element 18 or the second elongated element 20. In some embodiments, the first elongated element 18 and the second elongated element 20 may have the form of a braided or coiled cylinder, similar to a stent. The braided cylinder form of the first elongated element 18 may include a number of filaments 36 braided together to form a flexible first elongated element 18 which can be coupled to the tissue sample 22. The braided cylinder form of the first elongated element 18 may increase the surface area and planes of bonding between the tissue sample 22 and the first elongated element 22, greatly enhancing the coupling between the two components. Further, the braided cylinder shape may be ideal to allow the first elongated element 18 to deflect in response to a contraction of the tissue sample 22 and return to its original position once the tissue sample 22 has relaxed. In the field of intravenous medical devices, Nitinol stents are well known for their flexibility and strong bio-compatibility with biological tissue.

In some embodiments, it may be beneficial to maximize the surface area of the first elongated element 18 coupled to the tissue sample 22 in order to diffuse the electrical current passing through the tissue sample 22. For example, in embodiments where the first elongated element 18 is in contact with only a small area of the tissue sample 22, the electrical current passing from the first elongated element 18 to the tissue sample 22 may only pass into the tissue sample at that the small area, potentially causing the tissue sample 22 to be burned at that small area after repeated electrical stimulation. Alternatively, as shown in FIGS. 5 and 6, wherein the surface area of the first elongated element 18 in contact with the tissue sample 22 is maximized over a larger area, the electrical current may diffuse into the tissue sample 22 over that larger area, decreasing the chance that the tissue sample 22 may be burned.

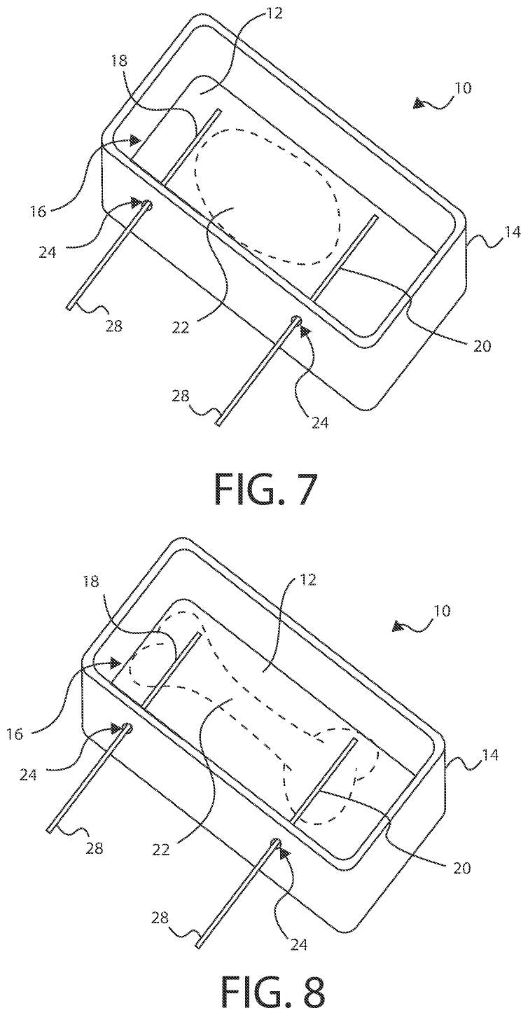

FIGS. 7, 8, and 9 show a series of perspective views of yet another embodiment of a tissue bioreactor, illustrating a process of using the tissue bioreactor. For example, FIG. 7 illustrates the tissue sample 22 may be placed within the chamber 16 and may initially be uncoupled to the first elongated element 18 and the second elongated element 20. A nutrition supplement (not shown) may also be placed within the chamber 16 to facilitate the growth of the tissue sample 22 within the chamber 16.

As illustrated by FIG. 8, after a set of conditions have been satisfied, the tissue sample 22 may grow to expand within the chamber 16, potentially growing onto the first elongated element 18 and the second elongated element 20. If the first elongated element 18 and the second elongated element 20 are made from bio-compatible material, the tissue sample 22 may chemically react to become coupled with the first elongated element 18 and the second elongated element 20. For example, if the first elongated element 18 were made of a nickel-titanium alloy, the surface coat of the first elongated element 18 may comprise a layer of titanium oxide, which may readily form chemical bonds with organic tissue samples 22. Once the first elongated element 18 and the second elongated element 20 are coupled to the tissue sample 22, movement of the tissue sample 22 within the chamber 16 will also causing movement and bending of the first elongated element 18 and the second elongated element 20.

In some embodiments, the first elongated element 18 and the second elongated element 20 may include a surface treatment such as roughening or cross-hatching to increase the coupling surface of the tissue sample 22 with the first elongated element 18 or the second elongated element 20. Additionally, the first elongated element 18 and the second elongated element 20 may have optimized cross-sectional shapes, such as being circular, ovoidal, or hexagonal, to increase the coupling surface between the tissue sample 22 and the first elongated element 18 or the second elongated element 20.

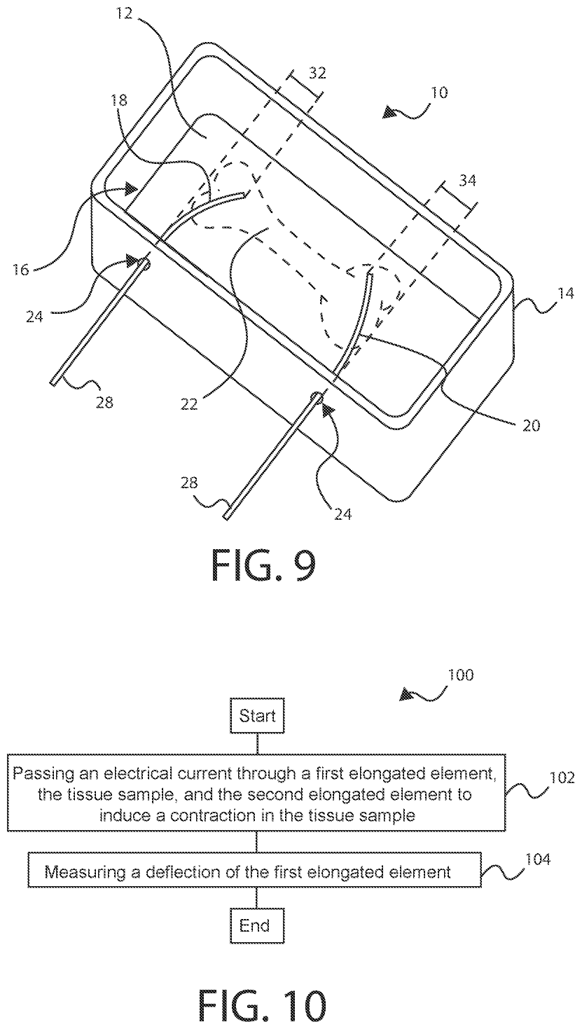

As illustrated in FIG. 9, the first elongated element 18, the tissue sample 22, and the second elongated element 20, the wires 28, and an electrical source (not shown) form at least a portion on electrical circuit. In such an arrangement, an electrical current may be passed from an electrical source (not shown) to one of the first elongated element 18 or the second elongated element 20, through the tissue sample 22, and then through the other of the first elongated element 18 and the second elongated element 20. Contraction of the tissue sample 22 may cause it to move to occupy less space within the chamber, thereby deflecting the first elongated element 18 and the second elongated element 20.

In some embodiments, the intensity of the contraction of the tissue sample 22 may be quantified by optically measuring the change in size of the tissue sample 22 itself. In other embodiments, the intensity of the contraction of the tissue sample 22 may be quantified by measuring the deflection 32 of the first elongated element 18 and the deflection 34 of the second elongated element 20. For example, the deflections 32, 34 may be measured by optically observing the first elongated element 18 and the second elongated element 20.

Alternatively, in other embodiments, the deflections 32, 34 of the first elongated element 18 and the second elongated element 20 may be measured using other methods. For example, as the first elongated element 18 deflects and bends from its resting state, the first elongated element 18 may stretch slightly and the cross-sectional area of the first elongated element 18 may become slightly smaller, thereby increasing electrical resistance. Therefore, the deflections 32, 34 of the first elongated element 18 and the second elongated element 20 may be measured by precisely measuring changes in resistance within the electrical circuit during the deflection.

In other embodiments, contraction of the tissue sample 22 may cause the first elongated element 18 and the second elongated element 20 to deflect toward one another, decreasing the distance between them. The electrical current passing through the first elongated element 18 and the second elongated element 20 may create a measurable magnetic field between first elongated element 18 and the second elongated element 20. As the distance between the first elongated element 18 and the second elongated element 20 decrease, the magnetic field strength between them may increase. Therefore, the deflections 32, 34 may be measured by observing a change in magnetic field strength between the first elongated element 18 and the second elongated element 20.

Furthermore, although specific components are described above, methods, systems, and articles of manufacture described herein may include additional, fewer, or different components. For example, some embodiments may have a third elongated element arranged in a different direction for measuring contraction along a different axis.

FIG. 10 illustrates a flow diagram of operations (100) to use the tissue bioreactor. The operations may include fewer, additional, or different operations than illustrated in FIG. 10. Alternatively, or in addition, the operations may be performed in a different order than illustrated.

The operation if using the tissue bioreactor (100) may include passing an electrical current through the first elongated element 18, the tissue sample 22, and the second elongated element 20 to induce a contraction of the tissue sample 22 (102). The method may further include measuring a deflection of a first elongated element 18 resulting from the contraction of the tissue sample 22 (104).

In addition to the advantages that have been described, it is also possible that there are still other advantages that are not currently recognized but which may become apparent at a later time. While various embodiments have been described, it will be apparent to those of ordinary skill in the art that many more embodiments and implementations are possible. Accordingly, the embodiments described herein are examples, not the only possible embodiments and implementations.

I claim:

1. A tissue bioreactor comprising:
a basin comprising a base and a sidewall, the base and the sidewall defining a chamber adapted to contain a tissue sample;
a first elongated element extending into the chamber of the basin, wherein the first elongated element is bio-compatible and electrically conductive; and
a second elongated element extending into the chamber, wherein the second elongated element is bio-compatible and is spaced apart from the first elongated element within the chamber.

2. The tissue bioreactor of claim 1, wherein the second elongated element is electrically conductive.

3. The tissue bioreactor of claim 1, wherein the first elongated element and the second elongated element extend into the chamber through a first side of the sidewall.

4. The tissue bioreactor of claim 3, wherein one of the first elongated element and the second elongated element extend across the chamber and are coupled to a second side of the sidewall.

5. The tissue bioreactor of claim 3, wherein one of the first elongated element and the second elongated element comprise an end positioned within the chamber and movement of the end within the chamber is unrestricted by the sidewall.

6. The tissue bioreactor of claim 1, wherein one of the first elongated element and the second elongated element extend into the chamber through the base.

7. The tissue bioreactor of claim 1, wherein the first elongated element is made of a nickel-titanium alloy.

8. The tissue bioreactor of claim 7, wherein the first elongated element has the shape of a coiled spring.

9. The tissue bioreactor of claim 7, wherein the first elongated element comprises a plurality of filaments braided together in a cylindrical shape.

10. A tissue testing apparatus, comprising:
a basin comprising a base and a sidewall, the base and the sidewall defining a chamber;
a tissue sample arranged within the chamber of the basin;
a first elongated element extending into the chamber of the basin, wherein the first elongated element is bio-compatible and electrically conductive; and
a second elongated element extending into the chamber, wherein the second elongated element is bio-compatible, is electrically conduct, and is spaced apart from the first elongated element within the chamber.

11. The tissue testing apparatus of claim 10, wherein the tissue sample is configured to grow onto the first elongated element and the second elongated element.

12. The tissue testing apparatus of claim 10, wherein the tissue sample is coupled to the first elongated element on a first side and coupled to the second elongated element on a second side.

13. The tissue testing apparatus of claim 12, wherein the first elongated element, the second elongated element, and the tissue sample comprise a portion of an electrical circuit.

14. The tissue testing apparatus of claim 12, wherein the first elongated element has a propensity to bending which is greater than the second elongated element.

15. The tissue testing apparatus of claim 14, wherein:
the first elongated element comprises an end positioned within the chamber wherein movement of the end within the chamber is unrestricted by the sidewall; and
the second elongated element extends across the chamber and is rigidly coupled to a second side of the sidewall.

16. The tissue testing apparatus of claim 14, wherein a thickness of the second elongated element is greater than a thickness of the first elongated element.

17. The tissue testing apparatus of claim 10, wherein the basin is made of a non-conductive, non-biocompatible material.

18. A method of using a tissue bioreactor comprising basin comprising a base and a sidewall, the base and the sidewall defining a chamber, a tissue sample positioned within the chamber, a first elongated element coupled to a first side of the tissue sample, and a second elongated element coupled to a second side of the tissue sample, the method comprising:
passing an electrical current through the first elongated element, the tissue sample, and the second elongated element to induce a contraction of the tissue sample; and
measuring a deflection of the first elongated element resulting from the contraction of the tissue sample.

19. The method of claim 18, wherein measuring the deflection of the first elongated element comprises observing a change in electrical resistance in during the deflection of the first elongated element.

20. The method of claim 18, wherein measuring the deflection of the first elongated element comprises observing a change in a magnetic field strength between the first elongated element and the second elongated element during the deflection of the first elongated element.
